(19) United States
(12) Patent Application Publication
(10) Pub. No.: US 2023/0056738 A1
(43) Pub. Date: Feb. 23, 2023

(54) EVALUATION METHOD FOR HYDROCARBON EXPULSION OF POST- TO OVER-MATURE MARINE SOURCE ROCKS

(71) Applicant: INSTITUTE OF GEOLOGY AND GEOPHYSICS, CHINESE ACADEMY OF SCIENCES, Beijing (CN)

(72) Inventors: Wenyang WANG, Beijing (CN); Yaping WANG, Beijing (CN); Xiongqi PANG, Beijing (CN); Zhangxing CHEN, Beijing (CN); Wang ZHANG, Beijing (CN); Rixiang ZHU, Beijing (CN)

(73) Assignee: INSTITUTE OF GEOLOGY AND GEOPHYSICS, CHINESE ACADEMY OF SCIENCES, Beijing (CN)

(21) Appl. No.: 17/530,487

(22) Filed: Nov. 19, 2021

(30) Foreign Application Priority Data
Aug. 20, 2021 (CN) 202110961245.0

(51) Int. Cl. G01N 25/22; G01N 33/24

(52) U.S. Cl. CPC G01N 25/22; G01N 33/241

(57) ABSTRACT

An evaluation method for hydrocarbon expulsion of post- to over-mature marine source rocks includes: establishing a hydrocarbon expulsion evolution profile of post- to over-mature source rocks; determining a critical condition for hydrocarbon expulsion from the source rocks, inverting original hydrocarbon generation potential of the source rocks, and establishing a hydrocarbon expulsion model for the source rocks; determining a hydrocarbon expulsion rate and cumulative hydrocarbon expulsion of the source rocks; and calculating hydrocarbon expulsion of the source rocks. The evaluation method establishes a hydrocarbon expulsion model for post- to over-mature source rocks without relying on immature to sub-mature samples. The evaluation method provides a scientific basis for the evaluation of the potential of deep oil and gas resources, and provides strong theoretical guidance and technical support for deep oil and gas exploration.

### CROSS REFERENCE TO THE RELATED APPLICATIONS

This application is based upon and claims priority to Chinese Patent Application No. 202110961245.0, filed on Aug. 20, 2021, the entire contents of which are incorporated herein by reference.

### TECHNICAL FIELD

The present disclosure belongs to the technical field of oil and gas exploitation, and in particular relates to an evaluation method for hydrocarbon expulsion of post- to over-mature marine source rocks.

### BACKGROUND

Hydrocarbon expulsion of source rocks is the most important subject for studying the hydrocarbon generation evolution of source rocks and the potential prediction of oil and gas resources, which is related to the basis of the decision-making on oil and gas exploration. However, the petroleum geology and geochemistry community has long faced a difficult problem, that is, how to establish a hydrocarbon generation and expulsion model of post- to over-mature marine source rocks and to calculate the hydrocarbon generation and expulsion of post- to over-mature marine source rocks. The fundamental reason for this long-standing problem lies in that the natural marine source rocks generally have a high maturity and there are few immature source rocks and sub-mature source rocks, which makes it hard to reconstruct a complete hydrocarbon generation evolution process of source rocks.

Scholars at home and abroad try to break through in two directions. First, sub-mature marine source rocks from relatively newer shallow strata in the basin are used to make up for the lack of samples in the study strata of post- to over-mature source rocks. According to the relationship between the total organic carbon (TOC) and hydrocarbon generation potential of the source rocks, the hydrocarbon generation and expulsion potential of post- to over-mature source rocks in deep strata are predicted. Second, sub-mature marine source rock samples from other basins are used to make up for the lack of samples in the study areas of post- to over-mature source rocks, and the hydrocarbon generation and expulsion of post-to over-mature source rocks are calculated by using the hydrocarbon generation potential method. At present, the industry professionals are focusing on searching for sub-mature source rock samples. However, ancient marine post- to over-mature source rocks in the study strata generally lack sub-mature source rocks, and no sub-mature source rocks have been found in China’s Lower Paleozoic marine strata. Therefore, it is problematic to use the immature or sub-mature marine source rocks from relatively newer shallow strata in the same or different basins for making up for the lack of sub-mature source rocks. There are great differences between strata in different basins and between sedimentary strata of different age in the same basin in terms of depositional environment, organic facies, organic matter types and organic matter enrichment conditions, which play an important role in the hydrocarbon generation evolution of source rocks. If the hydrocarbon expulsion characteristics of source rocks are not well understood, it is hard to scientifically predict the potential of oil and gas resources according to the genesis, which finally affects the scientific decision-making on the exploration strategies.

### SUMMARY

In order to solve the problem that the prior art cannot accurately and quantitatively evaluate the hydrocarbon expulsion of post- to over-mature source rocks, the present disclosure provides an evaluation method for hydrocarbon expulsion of post- to over-mature marine source rocks. The method includes the following steps: S100: establishing a hydrocarbon expulsion evolution profile of source rocks;

S200: determining a critical condition for hydrocarbon expulsion from the source rocks, inverting original hydrocarbon generation potential of the source rocks, and establishing a hydrocarbon expulsion model for the source rocks;

S300: determining a hydrocarbon expulsion rate and cumulative hydrocarbon expulsion of the source rocks; and

S400: calculating hydrocarbon expulsion of the source rocks.

In some preferred examples, the establishing a hydrocarbon expulsion evolution profile of source rocks may include: calculating a hydrocarbon generation potential index and an equivalent vitrinite reflectance based on a pyrolysis experiment of the source rocks; and

establishing a hydrocarbon expulsion evolution profile of the source rocks based on the hydrocarbon generation potential index and the equivalent vitrinite reflectance, where

the hydrocarbon generation potential index is 100 × (S₁ + S₂)/TOC, where S₁, S₂ are hydrocarbon yields per unit mass of source rock samples heated to 300° C. and 300-600° C. respectively, mg•HC/g; TOC is total organic carbon (TOC) per unit mass of the source rocks, mg/g; and the equivalent vitrinite reflectance is Rₒ, Rₒ = 0.0078Tₘₐₓ - 1.3654, where Tₘₐₓ is a maximum peak pyrolysis temperature in the pyrolysis experiment of the source rocks.

In some preferred examples, the determining a critical condition for hydrocarbon expulsion may include: obtaining a homogenization temperature distribution map of fluid inclusions according to an inclusion experiment;
determining a main peak value of a homogenization temperature for a first phase of the fluid inclusions based on the homogenization temperature distribution map of the fluid inclusions; and
obtaining a corresponding minimum Rₘᵢₙ of an isotherm at the main peak value of the homogenization temperature of the first phase of the inclusions according to a depositional burial history and a thermal evolution history of a typical well, where Rₘᵢₙ is a critical maturity Rₒₑ for hydrocarbon expulsion corresponding to the critical condition for hydrocarbon expulsion.

In some preferred examples, the inverting original hydrocarbon generation potential of the source rocks may include: obtaining a hydrocarbon generation potential index envelope according to the hydrocarbon expulsion evolution profile of the source rocks;
obtaining a fitted relation Ig based on the equivalent vitrinite reflectance and the hydrocarbon generation potential index envelope,
$$I g = \frac{a}{1 + {\text{e}}^{- \text{b} \left({\text{R}}_{\text{o}} - \text{c}\right)}} + \text{d,}$$
where a, b, c and d are constants; and
obtaining original hydrocarbon generation potential I_{og} of the source rocks based on the fitted relation and the critical maturity for hydrocarbon expulsion, I_{og} =
$$\frac{a}{1 + {\text{e}}^{- \text{b} \left({\text{R}}_{\text{oe}} - \text{c}\right)}} + \text{d} \text{.}$$
.

In some preferred examples, the establishing a hydrocarbon expulsion model for post- to over-mature source rocks may include: establishing a hydrocarbon expulsion model for post- to over-mature source rocks by means of matrix laboratory (MATLAB) based on the hydrocarbon expulsion evolution profile, the critical condition for hydrocarbon expulsion and the original hydrocarbon generation potential.

In some preferred examples, the determining a hydrocarbon expulsion rate and cumulative hydrocarbon expulsion of the source rocks may include: obtaining a hydrocarbon expulsion rate qₑ and cumulative hydrocarbon expulsion q_{ce} of the source rocks based on the hydrocarbon expulsion model for the source rocks, where

$${q}_{e} = {\text{I}}_{\text{og}} - I g .$$

$${q}_{c e} = \int {q}_{e} d \left({R}_{o}\right) .$$

In some preferred examples, the calculating hydrocarbon expulsion of the source rocks may include: obtaining a hydrocarbon expulsion intensity Iₑ of the source rocks in different thermal evolution stages according to an integral the hydrocarbon expulsion rate, abundance of organic matter and a thickness and density of the source rocks; and

obtaining total hydrocarbon expulsion Qₑ in each geological period based on the hydrocarbon expulsion intensity, where

$${I}_{e} = {\int }_{{R}_{o} {}^{t}}^{{R}_{o}} {10}^{- 3} ∗ {q}_{e} ∗ H ∗ ρ ∗ T O {C}_{O} ∗ d \left({R}_{o}\right)$$

$${Q}_{e} = {\int }_{{R}_{0} {}^{t}}^{{R}_{o}} {10}^{- 13} ∗ {q}_{e} ∗ H ∗ A ∗ ρ ∗ T O {C}_{O} ∗ d \left({R}_{o}\right)$$

where, H is the thickness of the source rocks; p is the density of the source rocks; A is a distribution area of the source rocks; and TOCₒ is original TOC of the source rock.

In some preferred examples, TOCₒ = TOC ∗ k; and

$$k = \left(1 - 0.83 ∗ \frac{\text{Ig}}{1000}\right) / \left(1 - 0.83 ∗ \frac{{\text{I}}_{\text{og}}}{1000}\right) .$$

1) The method of the present disclosure can establish a hydrocarbon expulsion model for post- to over-mature source rocks without relying on immature to sub-mature samples, which provides a reliable test model for the study of hydrocarbon expulsion characteristics of post- to over-mature source rocks.

2) The present disclosure forms a process for evaluating the hydrocarbon expulsion of post- to over-mature source rocks, which can scientifically calculate the hydrocarbon expulsion of source rocks in ancient marine strata lacking immature to sub-mature samples. Therefore, the present disclosure provides a scientific basis for the evaluation of the potential of deep oil and gas resources.

### BRIEF DESCRIPTION OF THE DRAWINGS

Other features, objectives and advantages of the present application will become more apparent upon reading the detailed description of the non-restrictive examples with reference to the following accompanying drawings.

FIG. 1 is a flowchart of a specific example of the present disclosure;

FIG. 2A is the variation of hydrocarbon generation potential of source rocks with thermal evolution, and FIG. 2B is the hydrocarbon expulsion rate of source rocks with thermal evolution;

FIG. 3 is a hydrocarbon generation potential evolution profile of Ediacaran algal dolomite source rocks in the Sichuan Basin, China;

FIG. 4 is a homogenization temperature distribution map of Ediacaran dolomite fluid inclusions in the Sichuan Basin;

FIG. 5 is a depositional burial history and a thermal evolution history of Well Moxi 8 in the Sichuan Basin;

FIG. 6 is a hydrocarbon expulsion model for the Ediacaran algal dolomite source rocks in the Sichuan Basin; and

FIG. 7 is a hydrocarbon expulsion intensity map of the Jurassic-Ediacaran algal dolomite source rocks in the Sichuan Basin.

### DETAILED DESCRIPTION OF THE EMBODIMENTS

The preferred implementations of the present disclosure are described below with reference to the accompanying drawings. Those skilled in the art should understand that the implementations herein are merely intended to explain the technical principles of the present disclosure, rather than to limit the protection scope of the present disclosure.

The present disclosure provides an evaluation method for hydrocarbon expulsion of post- to over-mature marine source rocks. The method includes the following steps. S 100: Establish a hydrocarbon expulsion evolution profile of post- to over-mature source rocks. Specifically, this step includes: calculate a hydrocarbon generation potential index and an equivalent vitrinite reflectance based on a pyrolysis experiment of the source rocks; and establish a hydrocarbon expulsion evolution profile of the source rocks based on the hydrocarbon generation potential index and the equivalent vitrinite reflectance, where the hydrocarbon generation potential index is 100 × (S₁ + S₂)/TOC , where S₁, S₂ are hydrocarbon yields per unit mass of source rock samples heated to 300° C. and 300-600° C. respectively, mg•HC/g; TOC is total organic carbon (TOC) per unit mass of the source rocks, mg/g; and the equivalent vitrinite reflectance is Rₒ, R_{O} = 0.0078Tₘₐₓ - 1.3654, where Tₘₐₓ is a maximum peak pyrolysis temperature in the pyrolysis experiment of the source rocks.

S200: Determine a critical condition for hydrocarbon expulsion, invert original hydrocarbon generation potential of the source rocks, and establish a hydrocarbon expulsion model for the source rocks. Specifically, the determining a critical condition for hydrocarbon expulsion includes: obtain a homogenization temperature distribution map of fluid inclusions according to an inclusion experiment of Dengying Formation; determine a main peak value of a homogenization temperature for a first phase of the fluid inclusions in the Dengying Formation based on the homogenization temperature distribution map of the fluid inclusions; and obtain a corresponding minimum Rₘᵢₙ of an isotherm at the main peak value of the homogenization temperature of the first phase of the inclusions according to a depositional burial history and a thermal evolution history of a typical well, where Rₒₑ is a critical maturity for hydrocarbon expulsion corresponding to the critical condition for hydrocarbon expulsion. The inverting original hydrocarbon generation potential of the source rocks includes: obtain a hydrocarbon generation potential index envelope according to the hydrocarbon expulsion evolution profile of the source rocks; obtain a fitted relation Ig based on the equivalent vitrinite reflectance and the hydrocarbon generation potential index envelope,

$$I g = \frac{a}{{\text{1+e}}^{- \text{b} \left({\text{R}}_{\text{o}} - \text{c}\right)}} \text{+d} ,$$

where a, b, c and d are constants; and obtain an original hydrocarbon generation potential I_{og} of the source rocks based on the fitted relation and the critical maturity for hydrocarbon expulsion,

$${I}_{o g} = \frac{a}{1 + {\text{e}}^{- \text{b} \left({\text{R}}_{\text{oe}} - \text{c}\right)}} + \text{d} \text{.}$$

The establishing a hydrocarbon expulsion model for post- to over-mature source rocks includes: establish a hydrocarbon expulsion model for post- to over-mature source rocks by means of matrix laboratory (MATLAB) based on the hydrocarbon expulsion evolution profile, the critical condition for hydrocarbon expulsion and the original hydrocarbon generation potential.

S300: Determine a hydrocarbon expulsion rate and cumulative hydrocarbon expulsion of the source rocks. Specifically, the determining a hydrocarbon expulsion rate and cumulative hydrocarbon expulsion of the source rocks includes: obtain a hydrocarbon expulsion rate qₑ and cumulative hydrocarbon expulsion q_{ce} of the source rocks based on the hydrocarbon expulsion model for the source rocks, where qₑ = I_{og} - Ig, q_{ce} = ∫ qₑd(Rₒ).

S400: Calculate hydrocarbon expulsion of the source rocks. Specifically, the calculating hydrocarbon expulsion of the source rocks includes: obtain a hydrocarbon expulsion intensity Iₑ of the source rocks in different thermal evolution stages according to an integral of the hydrocarbon expulsion rate, abundance of organic matter and a thickness and density of the source rocks in different thermal evolution stages; and obtain total hydrocarbon expulsion Qₑ in each geological period based on the hydrocarbon expulsion intensity, where

$${I}_{e} = {\int }_{{R}_{0} {}^{t}}^{{R}_{o}} {10}^{- 3} ∗ {q}_{e} ∗ H ∗ ρ ∗ T O {C}_{O} ∗ d \left({R}_{o}\right) ; \text{and}$$

$${Q}_{e} = {\int }_{{R}_{o} {}^{t}}^{{R}_{o}} {10}^{- 13} ∗ {q}_{e} ∗ H ∗ A ∗ p ∗ T O {C}_{O} ∗ d \left({R}_{o}\right) ;$$

where, H is the thickness of the source rocks; p is the density of the source rocks; A is a distribution area of the source rocks; and TOC_{O} is original TOC of the source rock.

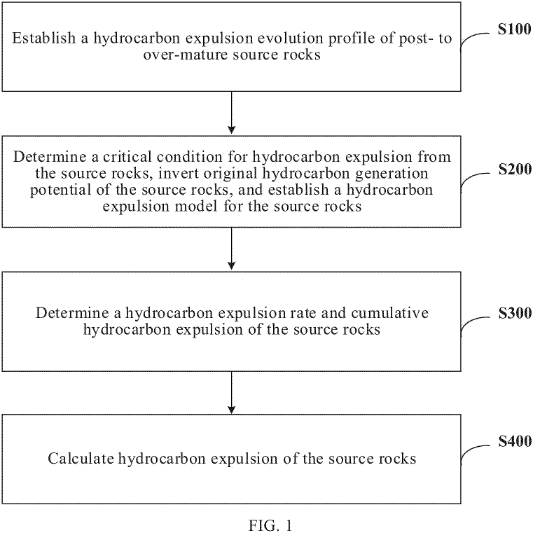

The present disclosure is described in further detail below with reference to FIGS. 1 to 7 and an example of the Sichuan Basin in China.

The Sichuan Basin is located in central China, with an area of about 19×10⁴ km², and it is one of the major natural gas producing areas in China. The Sichuan Basin is a typical superimposed petroliferous basin. After undergoing multi-cycle tectonic movements and the superimposition and transformation of multiple types of basins, the Sichuan Basin has formed multiple sets of source-reservoir-caprock assemblages, which have the characteristics of multi-layered hydrocarbon-bearing. The Ediacaran to Lower Triassic strata in the Sichuan Basin are marine carbonate strata, and the study strata of the present disclosure are in the Ediacaran Dengying Formation. According to lithology and biological characteristics, the Dengying Formation is divided into four lithological members from top to bottom, namely, Deng 4 (Z₂d⁴), Deng 3 (Z₂d³), Deng 2 (Z₂d²) and Deng 1 (Z₂d¹). Algal dolomite, which is widely distributed in the Sichuan Basin, is an important Ediacaran source rock in the Sichuan Basin. It is mainly distributed in the Deng 4 (Z₂d⁴) and Deng 2 (Z₂d²) members. This type of source rock has a buried depth of more than 5,000 m, and has reached the post- to over-mature thermal evolution stage, with a thickness of 300-1,350 m.

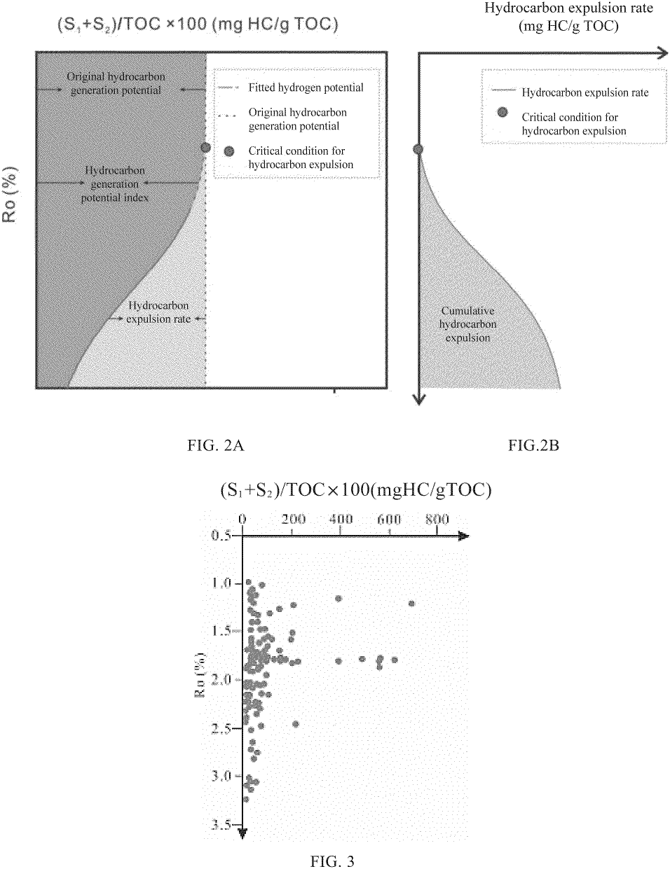

The present disclosure proposes an evaluation method for hydrocarbon expulsion of post- to over-mature marine source rocks. This method established a conceptual hydrocarbon expulsion model for post- to over-mature source rocks, as shown in FIGS. 2A and 2B, and was implemented by the following steps. A hydrocarbon generation potential evolution profile of the Ediacaran algal dolomite source rocks in the Sichuan Basin was established. According to parameters obtained from a pyrolysis experiment of the Ediacaran algal dolomite source rocks in the Sichuan Basin, a hydrocarbon generation potential index was calculated by 100×(S₁+S₂)/TOC. According to a pyrolysis parameter Tₘₐₓ, an equivalent vitrinite reflectance Rₒ (i.e., maturity) was calculated, and an evolution profile of 100×(S₁+S₂)/TOC changing with Rₒ, that is, a hydrocarbon expulsion evolution profile of the source rocks shown in FIG. 3 was plotted.

A critical condition for hydrocarbon expulsion from the Ediacaran algal dolomite source rocks in the Sichuan Basin was determined, original hydrocarbon generation potential of the source rocks was inverted, and a hydrocarbon expulsion model for the Ediacaran algal dolomite source rocks in the Sichuan Basin was established.

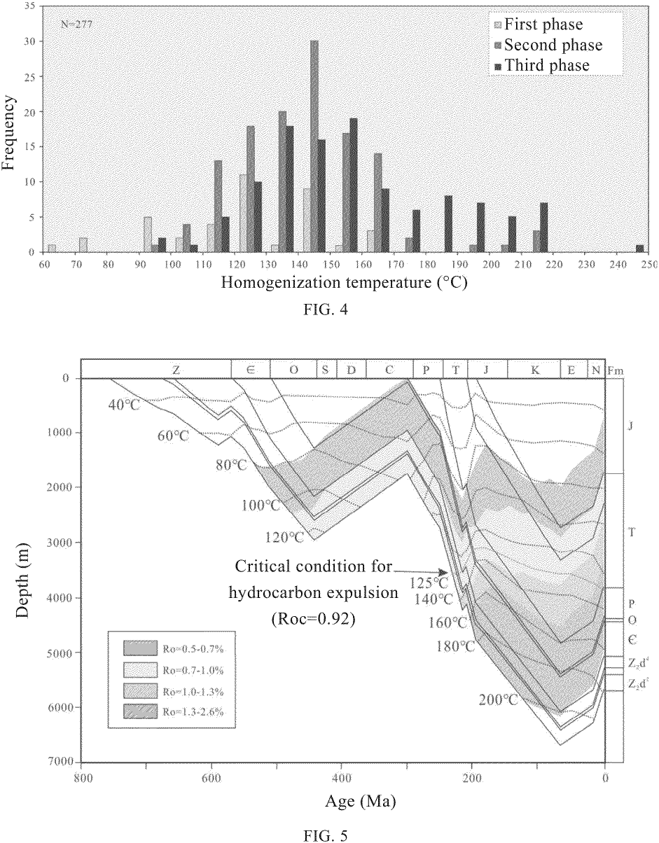

Through microscopic thin section analysis and geological analysis, three phases of inclusions were found in the Dengying Formation in the Sichuan Basin. The first phase of inclusions was formed in dolomite grains. Through experimental analysis of the inclusions in the Dengying Formation, a homogenization temperature distribution map of the fluid inclusions was obtained, as shown in FIG. 4. Based on the homogenization temperature distribution map of the fluid inclusions, a main peak value of the homogenization temperature of the first phase was determined. In this example, it was determined that the peak of the homogenization temperature of the inclusions in the first phase was 120-130° C. For quantitative characterization, 125° C. was taken as the main peak value of the homogenization temperature of the inclusions in the first phase, which meant that the source rocks began to expel a large amount of hydrocarbons at this paleo-geothermic temperature. According to a depositional burial history and a thermal evolution history of the typical Well Moxi 8 in the Sichuan Basin (FIG. 5), a critical maturity Rₒₑ for hydrocarbon expulsion from the algal dolomite source rocks in the Dengying Formation was inverted. The minimum Rₒ on the 125° C. isotherm of the Dengying Formation was the critical maturity for hydrocarbon expulsion from the algal dolomite source rocks of the Dengying Formation. Rₒ was taken as 0.92%, which meant that the Ediacaran algal dolomite source rocks in the Sichuan Basin began to expel a large amount of hydrocarbons when Rₒ reached 0.92%, that is, the critical maturity for hydrocarbon expulsion (Rₒₑ) was correspondingly Rₒₑ = 0.92%.

A hydrocarbon generation potential index envelope was obtained according to the hydrocarbon expulsion evolution profile of the source rocks, and a fitted relation Ig was obtained based on the equivalent vitrinite reflectance and the hydrocarbon generation potential index envelope. In this example,

$$I g = \frac{702.64}{1 + {\text{e}}^{- 2.17 \left({\text{R}}_{\text{o}} + 3.55\right)}} + 53.48.$$

The hydrocarbon generation potential corresponding to the critical maturity for hydrocarbon expulsion (Rₒₑ) on the hydrocarbon expulsion evolution profile of the source rocks was the original hydrocarbon generation potential of the source rocks. In this example, the corresponding original hydrocarbon generation potential of the Ediacaran algal dolomite source rocks in the Sichuan Basin was 756 mg•HC/g•TOC, that is I_{og} = 756 mg HC/g TOC.

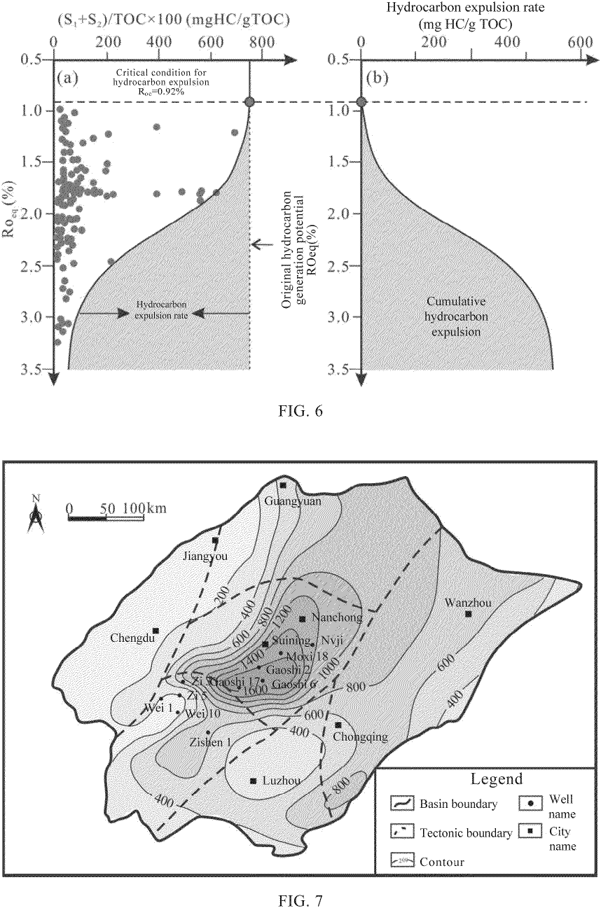

According to the determined hydrocarbon expulsion evolution profile, critical condition for hydrocarbon expulsion and original hydrocarbon generation potential, a hydrocarbon expulsion model for the Ediacaran algal dolomite source rocks in the Sichuan Basin was established (FIG. 6). In this model, the critical condition for hydrocarbon expulsion from the source rocks corresponded to the original hydrocarbon generation potential, and the hydrocarbon generation potential index of source rocks decreased with the increase of the thermal maturity.

Further, according to the established hydrocarbon expulsion model for the Ediacaran algal dolomite source rocks in the Sichuan Basin, a hydrocarbon expulsion rate qₑ and cumulative hydrocarbon expulsion q_{ce} of the algal dolomite were determined. qₑ was hydrocarbon expulsion per unit TOC of the source rocks at a certain degree of thermal evolution, and q_{ce} was cumulative hydrocarbon expulsion per gram of organic carbon from the source rocks.

Further,

$${q}_{e} = 702.52 - \frac{702.64}{1 + {\text{e}}^{- 2.17 \left({\text{R}}_{\text{o}} + 3.55\right)}} ;$$

where, q_{ce} = ∫ qₑd(Rₒ).

Further, the hydrocarbon expulsion from the Ediacaran algal dolomite source rocks in the Sichuan Basin was calculated. This step took the calculation of the hydrocarbon expulsion of the Jurassic-Ediacaran algal dolomite source rocks in the Sichuan Basin as an example. First, according to the integral of the hydrocarbon expulsion rate, abundance of organic matter and a thickness and density of the Jurassic-Ediacaran algal dolomite source rocks, a hydrocarbon expulsion intensity Iₑ of the Jurassic-Ediacaran algal dolomite source rocks was calculated. FIG. 7 shows the hydrocarbon expulsion intensity of the Jurassic-Ediacaran source rocks in the Sichuan Basin, which exceeded 1,600×10⁴ t/km² at a hydrocarbon expulsion center. Through the area integration of the hydrocarbon expulsion intensity Iₑ of the Jurassic-Ediacaran algal dolomite source rocks, the total hydrocarbon expulsion Qₑ of the Jurassic-Ediacaran algal dolomite source rocks was obtained.

$${I}_{e} = {\int }_{{R}_{0} {}^{t}}^{{R}_{o}} {10}^{- 3} ∗ {q}_{e} ∗ H ∗ A ∗ p ∗ T O {C}_{O} ∗ d \left({R}_{o}\right) .$$

$${Q}_{e} = {\int }_{{R}_{0} {}^{t}}^{{R}_{o}} {10}^{- 13} ∗ {q}_{e} ∗ H ∗ A ∗ p ∗ T O {C}_{O} ∗ d \left({R}_{o}\right) .$$

$$T O {C}_{o} = T O C ∗ k .$$

$$k = \left(1 - 0.83 ∗ \frac{\text{Ig}}{1000}\right) / \left(1 - 0.83 ∗ \frac{{\text{I}}_{\text{og}}}{1000}\right) .$$

The total hydrocarbon expulsion Qₑ of the Jurassic-Ediacaran algal dolomite source rocks in the Sichuan Basin was calculated to be 3958.4 × 10⁸ toe.

Although the present disclosure has been described with reference to the preferred examples, various improvements can be made and components therein can be replaced with equivalents without departing from the scope of the present disclosure. In particular, as long as there is no structural conflict, the technical features in the examples can be combined in any way. The present disclosure is not limited to the specific examples disclosed herein, but shall include all technical solutions falling within the scope of the claims.

In the description of the present disclosure, terms such as “central”, “upper”, “lower”, “left”, “right”, “vertical”, “horizontal”, “inner”, and “outer” indicate orientation or position relationships based on the accompanying drawings. They are merely intended to facilitate description, rather than to indicate or imply that the mentioned apparatus or components must have the specific orientation and must be constructed and operated in the specific orientation. Therefore, these terms should not be construed as a limitation to the present disclosure. Moreover, the terms such as “first”, “second”, and “third” are used only for description and are not intended to indicate or imply relative importance.

It should be noted that in the description of the present disclosure, unless otherwise clearly specified, meanings of terms “install”, “connect with” and “connect to” should be understood in a broad sense. For example, the connection may be a fixed connection, a removable connection, or an integral connection; may be a mechanical connection or an electrical connection; may be a direct connection or an indirect connection via a medium; or may be an internal connection between two assemblies. Those skilled in the art should understand the specific meanings of the above terms in the present disclosure based on specific situations.

In addition, terms “include”, “comprise”, or any other variations thereof are intended to cover non-exclusive inclusions, so that a process, an article, or a device/apparatus including a series of elements not only includes those elements, but also includes other elements that are not explicitly listed, or also includes inherent elements of the process, the article or the device/apparatus.

The technical solutions of the present disclosure are described with reference to the preferred implementations and drawings. Those skilled in the art should easily understand that the protection scope of the present disclosure is apparently not limited to these specific implementations. Those skilled in the art can make equivalent changes or substitutions to the relevant technical features without departing from the principles of the present disclosure, and the technical solutions derived by making these changes or substitutions should fall within the protection scope of the present disclosure.

1. A method of exploring a formation using an evaluation method for hydrocarbon expulsion of post- to over-mature marine source rocks comprises steps of:
A) collecting samples of post- to over-mature marine source rocks to be evaluated from a formation;
B) establishing a hydrocarbon expulsion evolution profile of the post- to over-mature source rocks comprises steps of:
calculating a hydrocarbon generation potential index and an equivalent vitrinite reflectance based on a pyrolysis experiment of the collected samples of post- to over-mature source rocks; and
establishing the hydrocarbon expulsion evolution profile of the post- to over-mature source rocks based on the hydrocarbon generation potential index and the equivalent vitrinite reflectance, wherein
the hydrocarbon generation potential index is
$$100 \times \left({\text{S}}_{1} + {\text{S}}_{2}\right) / T O C ,$$
wherein S₁, S₂ are hydrocarbon yields per unit mass of source rock samples heated to 300° C. and 300-600° C. respectively, mg•HC/g; TOC is total organic carbon (TOC) per unit mass of the post- to over-mature source rocks, mg/g; and the equivalent vitrinite reflectance is Rₒ, Rₒ = 0.0078Tₘₐₓ — 1.3654, wherein Tₘₐₓ is a maximum peak pyrolysis temperature in the pyrolysis experiment of the post- to over-mature source rocks;
C) determining a critical condition for the hydrocarbon expulsion of the post- to over-mature source rocks, inverting original hydrocarbon generation potential of the post- to over-mature source rocks, and establishing a hydrocarbon expulsion model for the post- to over-mature source rocks comprises steps of:
obtaining a homogenization temperature distribution map of fluid inclusions according to an inclusion experiment;
determining a main peak value of a homogenization temperature for a first phase of the fluid inclusions based on the homogenization temperature distribution map of the fluid inclusions; and
obtaining a corresponding minimum Rₘᵢₙ of an isotherm at the main peak value of the homogenization temperature of the first phase of the inclusions according to a depositional burial history and a thermal evolution history of a typical well, wherein Rₘᵢₙ is a critical maturity Rₒₑ for hydrocarbon expulsion corresponding to the critical condition for hydrocarbon expulsion;
wherein determining of inverting the original hydrocarbon generation potential of the post- to over-mature source rocks comprises:
obtaining a hydrocarbon generation potential index envelope according to the hydrocarbon expulsion evolution profile of the post- to over-mature source rocks;
obtaining a fitted relation Ig based on the equivalent vitrinite reflectance and the hydrocarbon generation potential index envelope by the following equation:
Ig = _{H}e_{-:CRo}-_{C}) + d, wherein a, b, c and d are constants; and
obtaining the original hydrocarbon generation potential I_{og} of the post- to over-mature source rocks based on the fitted relation and the critical maturity for hydrocarbon expulsion by the following equation:
$${I}_{o g} = \frac{a}{1 + {\text{e}}^{- \text{b} \left({\text{R}}_{\text{oe}} - \text{c}\right)}} + \text{d;}$$
D) determining a hydrocarbon expulsion rate and cumulative hydrocarbon expulsion of the post- to over-mature source rocks;
E) calculating the hydrocarbon expulsion of the post- to over-mature source rocks based on steps A) through D); and
F) wherein when the calculated hydrocarbon expulsion of the collected sample post- to over-mature source rocks to be evaluated of the formation meets a predetermined threshold, exploring the formation.

2. The method according to claim 1, wherein establishing the hydrocarbon expulsion model for the post- to over-mature source rocks comprises:
establishing the hydrocarbon expulsion model for the post- to over-mature source rocks by means of matrix laboratory (MATLAB) based on the hydrocarbon expulsion evolution profile, the critical condition for hydrocarbon expulsion and the original hydrocarbon generation potential.

3. The method according to claim 2, wherein determining the hydrocarbon expulsion rate and the cumulative hydrocarbon expulsion of the post- to over-mature source rocks comprises:
obtaining the hydrocarbon expulsion rate qₑ and the cumulative hydrocarbon expulsion q_{ce} of the post- to over-mature source rocks based on the hydrocarbon expulsion model for the post-to over-mature source rocks by the following equations:
$${q}_{e} = {I}_{o g} - I g ;$$
$${q}_{c e} = \int {q}_{e} d \left({R}_{o}\right)$$
.

4. The method according to claim 3, wherein calculating the hydrocarbon expulsion of the post- to over-mature source rocks comprises:
obtaining a hydrocarbon expulsion intensity Iₑ of the post- to over-mature source rocks in different thermal evolution stages by an integral of the hydrocarbon expulsion rate, abundance of organic matter and a thickness and density of the post- to over-mature source rocks corresponding to the different thermal evolution stages by the following equation:
$${I}_{e} = \int \begin{matrix}{}_{{R}_{o}} \\ {}^{{R}_{0}}\end{matrix} {}_{t} {10}^{- 3} * {q}_{e} * H * ρ * T O {C}_{o} * d ( {R}_{o} ) ;$$
and
obtaining total hydrocarbon expulsion Qₑ in each geological period based on the hydrocarbon expulsion intensity by the following equation:
$${Q}_{e} = {\int }_{{R}_{0}}^{{R}_{o}} {}_{t} {10}^{- 13} ∗ {q}_{e} ∗ H ∗ A ∗ ρ ∗ T O {C}_{O} ∗ d \left({R}_{o}\right) ;$$
wherein, H is the thickness of the post- to over-mature source rocks; p is the density of the post- to over-mature source rocks; A is a distribution area of the post- to over-mature source rocks; and TOC_{O} is original TOC of the post- to over-mature source rocks.

5. The method according to claim 4, wherein
TOCₒ = TOC * k; and
$$k = \left(1 - 0.83 ∗ \frac{I g}{1000}\right) / \left(1 - 0.83 ∗ \frac{{I}_{o g}}{1000}\right)$$
.
